(19) United States
(12) Patent Application Publication
(10) Pub. No.: US 2023/0338744 A1
(43) Pub. Date: Oct. 26, 2023

(54) IMPLANTABLE DEVICE FOR ORGAN OPERATION MODULATION

(71) Applicants: University of Cincinnati, Cincinnati, OH (US); Vanderbilt University, Nashville, TN (US)

(72) Inventors: Deeptankar DeMazumder, Baltimore, MD (US); Swati Dey, Nashville, TN (US)

(73) Assignees: University of Cincinnati, Cincinnati, OH (US); Vanderbilt University, Nashville, TN (US)

(21) Appl. No.: 18/009,449

(22) PCT Filed: Jun. 9, 2021
(86) PCT No.: PCT/US2021/036633

§ 371 Date: Dec. 9, 2022

Related U.S. Application Data
(60) Provisional application No. 63/036,510, filed on Jun. 9, 2020.

(51) Int. Cl. A61N 5/06

(52) U.S. Cl. CPC A61N 5/0622; A61N 5/0601; A61N 2005/0626; A61N 2005/0663; A61N 2005/0632

(57) ABSTRACT

A system for modulating operation of an organ in real time by controlling illumination of one or more light components is provided. The system includes an external device comprising a processing unit and a power supply configured to transmit stimulation parameters, a wireless implantable device comprising, a sensor configured to detect, in real time, activity data from a tissue cluster of an organ, a stimulator including a plurality of light components corresponding to at least a first wavelength and a second wavelength and a flexible elastomer coupled to the plurality of light components, and a transceiver configured to transmit the activity data to the external device, wherein the stimulator is configured to illuminate, based on the stimulation parameters, one of the plurality of light components coupled to the flexible elastomer, wherein the processing unit is configured to update the stimulation parameters based on the activity data.

### CROSS REFERENCE TO RELATED APPLICATION

This application claims benefit of U.S. Provisional Application No. 63/036,510 filed on Jun. 9, 2020, the entire contents of which are herein incorporated by reference.

### STATEMENT REGARDING FEDERALLY SPONSORED RESEARCH OR DEVELOPMENT

This invention was made with government support under PR182372 and PR191442 awarded by the Department of Defense and 4R00HK130662 awarded by the National Institutes of Health. The government has certain rights in the invention.

### TECHNICAL FIELD

The embodiments described herein generally relate to modifying the operation of an organ, and more specifically, to modulating the operation of an organ using an implantable device by illuminating one or more light components included in the device.

### BACKGROUND

One of the biggest challenges faced by the medical industry is effectively addressing and reducing risks of adverse health conditions without requiring active participation from doctors, first responders, and various other medical personnel. Various devices are currently available in the market that may be surgically embedded within humans and be configured to permanently activate or inhibit operation of one or more organs. However, there are currently no devices in the market that control operation of one or more organs across a range of levels based on data that is gathered, in real time, from these organs.

Accordingly, a need exists for an implantable device that modulates or controls operation of an organ based on real-time data regarding the functioning of the organ.

### SUMMARY

In one embodiment, a system for modulating operation of an organ in real time by controlling illumination of one or more light components is provided. The system includes an external device comprising a processing unit and a power supply configured to transmit stimulation parameters, a wireless implantable device comprising, a sensor configured to detect, in real time, activity data from a tissue cluster of an organ, a stimulator including a plurality of light components corresponding to at least a first wavelength and a second wavelength and a flexible elastomer coupled to the plurality of light components, and a transceiver configured to transmit the activity data to the external device, wherein the stimulator is configured to illuminate, based on the stimulation parameters, one of the plurality of light components coupled to the flexible elastomer, wherein the processing unit is configured to update the stimulation parameters based on the activity data.

In another embodiment, a method for modulating operation of an organ in real time by controlling illumination of one or more light components is provided. The method includes detecting, in real time, activity data from a tissue cluster of an organ, transmitting the activity data to an external device, receiving, from the external device, stimulation parameters for illumination of at least one of a plurality of light components, the plurality of light components coupled to a flexible elastomer, and illuminating, based on the stimulation parameters, at least one of the plurality of light components.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1A depicts non-limiting components of a system in which the implantable device of the present disclosure operates, according to one or more embodiments described and illustrated herein;

FIG. 1B depicts an example design of the implantable device of the present disclosure, according to one or more embodiments described and illustrated herein;

FIG. 2 depicts a flowchart for modulating an operation of an organ using the implantable device that operates as part of the system as described in the present disclosure, according to one or more embodiments described and illustrated herein;

FIG. 3 depicts graphical representations of fluorescence intensity profiles of a plurality of molecules, according to one or more embodiments described and illustrated herein;

FIG. 4 depicts additional graphical representations of fluorescence intensity profiles of a plurality of molecules, according to one or more embodiments described and illustrated herein;

FIG. 5 depicts graphical representations of fluorescence intensity profiles of a plurality of light components, according to one or more embodiments described and illustrated herein; and

FIG. 6 depicts an example flow chart including various steps for modulating sympathetic and parasympathetic nerves in order to reduce the risk of cardiac arrest while optimizing cardiac operation, according to one or more embodiments described and illustrated herein; and

FIG. 7 depicts an example flow chart including operation of an artificial intelligence engine that is utilized for modulating sympathetic and parasympathetic nerves in order to reduce the risk of cardiac arrest while optimizing cardiac operation, according to one or more embodiments described and illustrated herein.

### DETAILED DESCRIPTION

The embodiments disclosed herein describe an implantable device that may be surgically embedded adjacent to a nerve cluster, e.g., stellate ganglia, and be configured to activate or inhibit the operation of the nerve cluster by illuminating various light components, each of which may emit light corresponding to a particular wavelength. As stated above, while there are various medical devices in the market that may be embedded within human beings and be configured to permanently activate or inhibit operation of one or more organs, there are currently no devices in the market that control operation of one or more organs across a range of levels based on data that is gathered, in real time, from these organs. Additionally, current devices that are embedded in human beings also fail to analyze real-time data gathered regarding one or more organs using an artificial intelligence based software and controls operation of the embedded device using stimulation parameters generated by the artificial intelligence based software. The current implantable device addresses and overcomes these deficiencies.

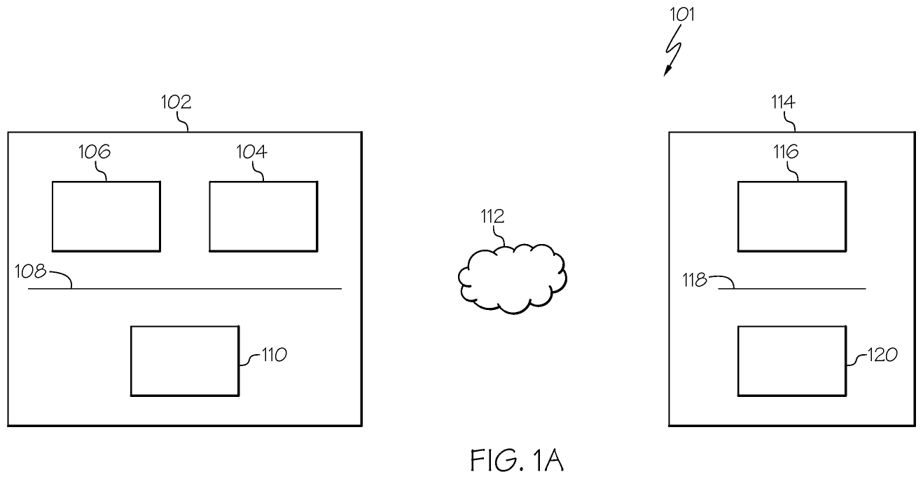

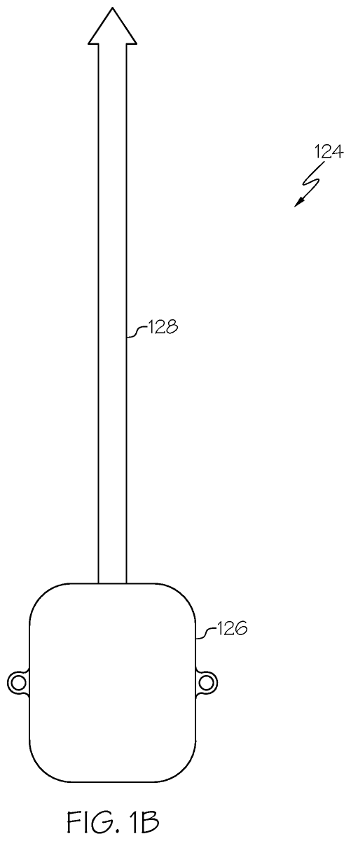

FIG. 1A depicts non-limiting components of a system in which the implantable device of the present disclosure operates, according to one or more embodiments described and illustrated herein.

Notably, FIG. 1A depicts a system 101 that includes an implantable device 102, and an external control device 114, and a communication network 112. In embodiments, the implantable device 102 may include a sensor 106, a stimulator 104, and an antenna 110. Each of these components may be communicatively coupled to one another via the communication path 108. The communication path 108 may be formed from any medium that is capable of transmitting a signal such as, for example, conductive wires, conductive traces, optical waveguides, or the like. In one embodiment, the communication path 108 may comprise a combination of conductive traces, conductive wires, connectors, and buses that cooperate to permit the transmission of electrical data signals to components such as processors, memories, sensors, input devices, output devices, and communication devices.

In embodiments, the implantable device 102 may be surgically implanted within a certain vicinity of, e.g., one or more organs of a human being. Specifically, in embodiments, portions of the implantable device 102 may be implanted adjacent to the stellate ganglia within a human body. The stellate ganglia are a collection of nerves located at the sixth and seventh cervical vertebrae. When the human body undergoes acute stress, the stellate ganglia (sympathetic nerves) stimulate B-adrenergic receptors for adaptive positive inotropy. Positive inotropy refers to the strengthening of the force of heart contractions. It is noted that a significant number of the sympathetic nerves that are utilized to provide sympathetic input to the heart originate from the stellate ganglia. Sympathetic input or stimulation increases heart rate and myocardial contractility, which may occur, for example, during exercise, emotional excitement, or under various pathological conditions (e.g., cardiac arrest). As such, modulating the operation of the stellate ganglia enables for the prevention of various heart related illnesses, e.g., heart failure, ventricular tachyarrhythmia, sudden cardiac arrest (SCD), and so forth.

In embodiments, the implantable device 102 may be formed of a flexible printed circuit board (PCB) from which a flexible elastomer (not shown) may protrude. In embodiments, the flexible PCB may have the dimensions of, e.g., 1 mm×14 mm, and the flexible elastomer may a length of 100 mm. It is noted that other dimensions of the PCB and the flexible elastomer are also contemplated. In embodiments, the flexible elastomer may be secured around the stellate ganglia with a nerve cuff (e.g., nerve cuff electrodes). In embodiments, the flexible elastomer may be attached to a particular tissue of interest (e.g., stellate ganglia, other tissues of organs) with the use of sutures. It is noted that the flexible elastomer of the implantable device 102 may include multiple portions (e.g., multiple channels) such that each portion or channel is designated for a particular light component (e.g., LED), which is configured to illuminate light in a particular wavelength. In embodiments, a first channel (e.g., a first portion) may be utilized by a first light component (e.g., a light emitting diode) to emit light (e.g., a first light) in a wavelength of 488 nanometers and a second channel (e.g., a second portion) may be utilized by a second light component (e.g., an additional light emitting diode) to emit light (e.g., a second light) in a wavelength of 405 nanometers. A plurality of additional channels designated for additional light components may also be included as part of the flexible elastomer. It is noted that the various channels may be configured to emit light, in real time, in different combinations, simultaneously, and so forth.

The sensor 106, the stimulator 104, and the antenna 110 may be embedded within or installed on various portions of the PCB. The sensor 106 is configured to detect electrical signals (e.g., ECG, cardiac output, etc.) emanating from various organs, nerves, and so forth. Specifically, the sensor 106 as described in the present disclosure is configured to detect fluorescence (based on the electrical signals emanating from nerves associated with various organs). In embodiments, the sensor 106 may be configured to detect, real time, one or more fluorescence values or fluorescence data, e.g., auto-fluorescence that may be emanating from enzymes such as nitrite reductase (NAD(P)/H) or proteins such as Green Fluorescent Protein (GFP). For example, the sensor 106 may be a photodiode configured to sense green roGFP fluorescence at 520 nanometers. The fluorescence (e.g., fluorescence values) of other enzymes, proteins, arachidonic acids, vitamins (Vitamin A), Flavins, and chemical compounds (e.g., PPIX) may also be detected by the sensor 106. The fluorescence values, which are based on electrical signals emanating from enzymes, proteins, chemical compounds, etc., may be routed by the sensor 106 to the antenna 110 via the communication path 108. Thereafter, the antenna 110 may communicate data gathered from the electrical signals (on which the fluorescence values are based) to the external device 114 via the communication network 112. It is noted that, in embodiments, an amplifier (not shown) may be installed as part of the implantable device 102 and serve to amplify the electrical signal detected by the sensor 106.

Additionally, the implantable device 102 may be utilized to identify regulations of the sympathetic nervous system (SNS), which trigger sudden cardiac death (SCD). It is noted that a vast majority (e.g., 90%) of the sympathetic nervous system input to a particular organ such as the heart originates from the nerve cluster refereed to as the Stellate Ganglia. Stress-induced hyperactivity of the stellate ganglia increases free radicals in the heart, which leads to sudden cardiac death (SCD).

In embodiments, the external control device 114 may analyze the received electrical signals using one or more processors 116. The one or more processors 116 may be any device capable of executing machine readable and executable instructions. Accordingly, the one or more processors 116 may be a controller, an integrated circuit, a microchip, a computer, or any other computing device. The analysis may also be performed using an artificial intelligence trained model. Based on the analysis, the external device 114 may route various stimulation parameters via the communication path 118 to the network interface 120. The network interface 120 may communicate the stimulation parameters to the implantable device 102 via the communication network 112. Upon receipt, the stimulation parameters may be routed via the communication path 108 to the stimulator 104. In accordance with the stimulation parameters, the stimulator 104 may activate a light component (e.g., an LED) such that the light component emits light corresponding to a particular wavelength, e.g., 405 nanometers, 488 nanometers, etc. It is noted, in embodiments, multiple LEDs may also be activated.

FIG. 1B depicts an example design 124 of the implantable device 102 of the present disclosure, according to one or more embodiments described and illustrated herein. Specifically, the example design 124 includes a flexible PCB 126 on which each of the stimulator 104, the sensor 106, and the antenna 110 may be positioned. A flexible elastomer 128 may be adhered to and protrude from a portion of the flexible PCB 126 and be configured around a set of nerves, e.g., stellate ganglia. Additionally, as previously stated, the flexible elastomer 128 may include a plurality of portions or channels, each of which may be dedicated for a particular light component that emits light at a distinct wavelength. For example, the flexible elastomer 128 may include a first channel designated for a light component that emits light at a wavelength of 488 nanometers and a second channel designated for another light component that emits light at a wavelength of 405 nanometers.

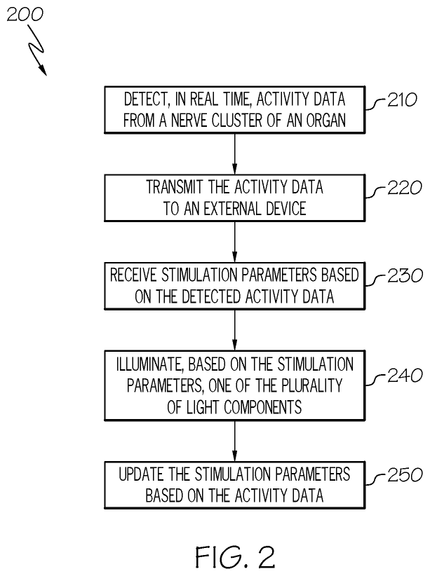

FIG. 2 depicts a flowchart 200 for modulating an operation of an organ using the implantable device 102, which operates as part of the system 101 as described in the present disclosure, according to one or more embodiments described and illustrated herein. In an example operation of the implantable device 102, the implantable device 102 may be surgically implanted with a human body and within a certain proximity of a nerve cluster, e.g., stellate ganglia. Additionally, in embodiments, genes may be transferred into the stellate ganglia for the purpose of expressing new protein chimeras. These chimeras will respond to the illumination of specific light components (LEDs) by exciting or inhibiting the operation of the nerve cluster or tissue cluster. The implantable device 102 may be positioned near a nerve or tissue cluster of various other organs within the human body. It is also noted that the implantable device 102 may communicate with one or more external devices (e.g., the external control device 114).

In block 210, the sensor 106 of the implantable device 102 may detect activity data from a tissue cluster, a nerve cluster within the human body. The activity data may include electrical signals (e.g., ECG, cardiac output), fluorescence values or fluorescence data. In embodiments, as stated above, such a nerve cluster may be stellate ganglia, which serves to provide sympathetic input to the heart. Such sympathetic input causes or results in an increased heart rate and myocardial contractility. Specifically, during periods of acute stress experienced by the human body, the stellate ganglia may stimulate beta adrenergic receptors on cardiac myocytes for adaptive positive inotropy, which results in an increased heart rate and myocardial contractility. In instances where the human body experiences prolonged stress and the operation of the stellate ganglia is not moderated, individuals may experience serious illnesses, e.g., heart failure, ventricular tachyarrhythmias, and sudden cardiac arrest. Other illnesses may also occur. Additionally, the sensor 106 may also detect or measure free radicals in various organs (e.g., the heart) in real time. Data relating to the free radicals may be included in the activity data.

As part of detecting, in real time, activity data from a tissue cluster or a nerve cluster, the sensor 106 may be configured to detect the generation of electrical signals by the nerve or tissue cluster (e.g., the stellate ganglia). The electrical signals that are detected are indicative of an auto-fluorescence value associated with the nerve cluster. Additionally, the sensor 106 may detect an additional auto-fluorescence value associated with one of a plurality of molecules. Specifically, the sensor 106 may detect auto-fluorescence values associated with enzymes, proteins, arachidonic acids, vitamins (Vitamin A), Flavins, and chemical compounds (PPIX). The detected auto-fluorescence values, which are based on the electrical signals from the nerve cluster (e.g., stellate ganglia), are included as part of the activity data of the nerve cluster.

In block 220, after gathering the activity data, the sensor 106 may route the activity data to the antenna 110 via the communication path 108. In embodiments, the antenna 110 may wirelessly communicate the activity data to one or more external devices such as, e.g., the external control device 114. Upon receiving the activity data, the external control device 114 may analyze the activity data, in real time, using the one or more processors 116. In embodiments, the external control device 114 may be a desktop computing device, a laptop, a server, or a combination thereof.

The analysis of the activity data, in real time, by the external control device 114, may include comparing the fluorescence or auto-fluorescence values associated with proteins, enzymes, and so forth, with certain threshold values. For performing the analysis, the external control device 114 may utilize one or more software applications that may include an artificial intelligence based neural network trained model. The analysis may also involve the external control device 114 accessing one or more databases that includes historical data associated with the fluorescence levels of various organs, nerve clusters, tissue clusters etc. Additionally, the historical data may be gathered over various time frames, under various conditions, in various individuals, across genders, etc. Additionally, the analysis may involve the external control device 114 accessing, cataloging, and analyzing historical responses and outcomes for a particular individual, a particular subgroup (e.g., ethnicity, gender, age, etc.), etc., based on certain levels of fluorescence, auto-fluorescence, and the like. Based on the analysis, the external control device 114 may generate one or more stimulation parameters. It is noted that the fluorescence and/or auto-fluorescence values may be utilized by the external control device 114 to determine a quantity of reactive oxygen species associated with one or more cells of the nerve or tissue cluster. It is further noted that the additional fluorescence and/or auto-fluorescence values may be associated with at least one of a plurality of molecules including proteins, arachidonic acid, and flavins, among other proteins, enzymes, and acids. The stimulation parameters may include instructions for illumination of at least one of the plurality of light components included in the implantable device 102, namely at least one of the plurality of light components that are included in or embedded in channels of the flexible elastomer 128. In embodiments, stimulation parameters are generated such that these parameters are specific to the characteristics of a particular operation of an organ within a human being. The stimulation parameters may include frequency, intensity, duration, duty cycle, and various other metrics directed to the illumination of one or more of the plurality of the light components. It is noted that the artificial intelligence based software generates parameters that are designed to either inhibit the operation of the stellate ganglia or activate the operation of stellate ganglia, in accordance to various circumstances.

For example, if the analysis of the activity data indicates that an organ (e.g., heart) of an individual is at a high risk, the external control device 114 may generate stimulation parameters that instruct one or more light components to be illuminated, which causes an inhibition or reduction in the activity of the stellate ganglia. In contrast, if the analysis of the activity data indicates that an organ (e.g., heart) of an individual is at low risk, but the body activity (e.g., respiration rate) and temperature are higher than a threshold level, the external device 114 may generate stimulation parameters for instructing one or more light components to be illuminated, which causes an increase in the activity of the stellate ganglia. The generated stimulation parameters may be communicated by the network interface 120 of the external control device 114 to the implantable device 102 via the communication network 112.

In block 230, the antenna 110 of the implantable device 102 may receive, from the external control device 114 (e.g., an external device), stimulation parameters for illumination of at least one of the plurality of light components. Upon receipt of the parameters, these parameters may be routed from the antenna 110 to the stimulator 104 via the communication path 108. As previously stated, each of the plurality of light components are coupled to the flexible elastomer 128 (e.g., embedded in various channels of the flexible elastomer 128).

In block 240, the stimulator 104 may illuminate, based on the stimulation parameters, at least one of the plurality of light components. As previously stated, if the analysis as described under block 220, indicates that an organ (e.g., heart) of an individual is at a high risk, the implantable device 102 may illuminate a light component corresponding to a wavelength that causes inhibition in the activity of the nerve or tissue cluster, e.g., stellate ganglia. Alternatively, the implantable device 102 may illuminate a light component corresponding to a wavelength that causes an increase in the activity of the nerve or tissue cluster, e.g., stellate ganglia. In embodiments, as described above, one or more light components may be illuminated, which in turn may cause new protein chimeras to respond to specific LEDs by activating or inhibiting a nerve cluster or tissue cluster (e.g., Stellate ganglia). Additionally, the activation or inhibition of the nerve cluster or tissue cluster will result in the reduction or modulation in levels of free radicals of an organ (e.g., the heart), which in turn reduces the likelihood of harmful diseases such as, e.g., heart failure, ventricular tachyarrhythmia, sudden cardiac arrest, and so forth. It is further noted that illumination of various light components results in the graded (modulated) activation or inhibition of sympathetic nervous system (SNS) activity (e.g., operation of the stellate ganglia).

In block 250, the external control device may update a set of stimulation parameters based on the activity data that was received. For example, the generated stimulation parameters may be stored in a database in association with one or more organs of various individuals. In embodiments, after a particular one of the plurality of light components are illuminated, and the operation of the stellate ganglia is inhibited or increased, the sensor 106 may detect additional auto-fluorescence values that may be communicated to the external control device 114. The external control device 114 may then perform additional analysis, and update an existing set of stimulation parameters or generate new stimulation parameters based on a changed condition of the nerve cluster or tissue cluster. The updating of existing stimulation parameters or generation of new stimulation parameters will result in the progressive tailoring of the stimulation parameters to specific organs of individuals and various subgroups of individuals. It is further noted that the updating of existing stimulation parameters or generation of new stimulation parameters involves comparing the activity data detected by the sensor 106 with historical data (e.g., historical responses and events associated with organs of individuals and various subgroups of various individuals), by the external control device 114.

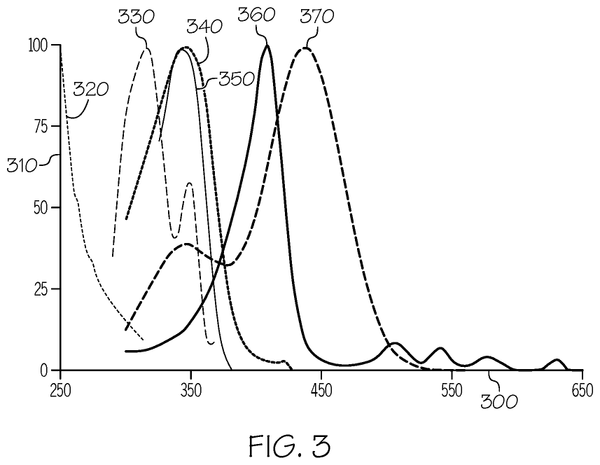

FIG. 3 depicts graphical representations of fluorescence intensity profiles of a plurality of molecules, according to one or more embodiments described and illustrated herein. As illustrated, an x-axis 300 corresponds to wavelength values (expressed in nanometers) and a y-axis 310 corresponds to fluorescence intensity values (expressed in au—relative emission intensity as arbitrary units). Graphical representation 320 depicts the fluorescence intensity profile of proteins across various wavelengths, a graphical representation 330 depicts the fluorescence intensity profile of Arachidonic acid across various wavelengths, and a graphical representation 340 depicts the fluorescence intensity profile of Vitamin A across various wavelengths across various wavelengths. Additionally, graphical representations 350, 360, and 370 represent fluorescence intensity profiles of NAD(P)H, PPIX, and Flavins respectively, across various wavelengths.

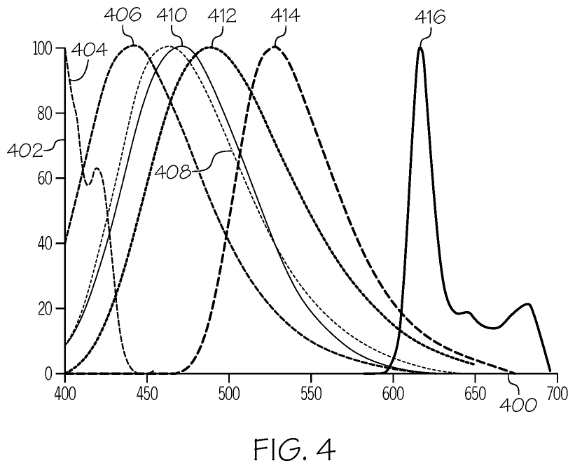

FIG. 4 depicts additional graphical representations of fluorescence intensity profiles of a plurality of molecules, according to one or more embodiments described and illustrated herein. As illustrated, an x-axis 400 corresponds to wavelength values (expressed in nanometers) and a y-axis 402 that corresponds to fluorescence intensity values (au). Graphical representation 404 depicts the fluorescence intensity profile of proteins, while graphical representations 406 and 408 depict the fluorescence intensity profiles of NAD(P)H (bound) and NAD(P)H (free). Additionally, graphical representations 410, 412, 414, and 416 depict the fluorescence intensity profiles of Arachidonic acid, Vitamin A, Flavins, and PPIX enzymes, respectively, across various wavelength values.

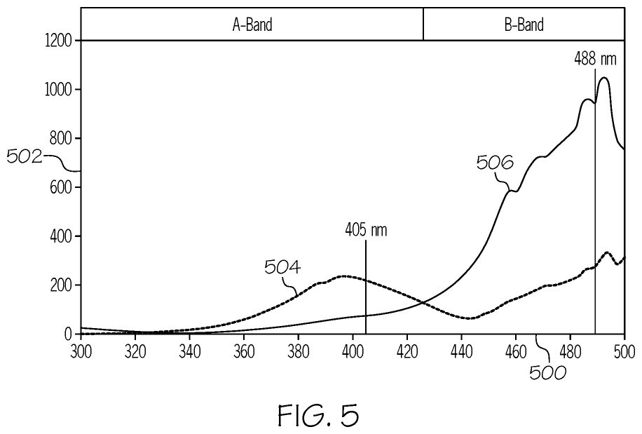

FIG. 5 depicts a graphical representations of the fluorescence intensity profiles of a plurality of light components, according to one or more embodiments described and illustrated herein. As illustrated, an x-axis 500 corresponds to wavelength values (expressed in nanometers) and a y-axis 502 corresponds to fluorescence intensity values (au). Graphical representation 504 depicts a fluorescence intensity profile of a light component that emits light having a wavelength of 405 nanometers and graphical representation 506 depicts a fluorescence intensity profile of a light component that emits light having a wavelength of 488 nanometers. As stated above, it is noted that illumination of the light components emitting light in the wavelengths of 405 and 488, based on analysis of the activity data, may result in the inhibition or activation (excitation) of the activity of the nerve or tissue clusters of an organ (e.g., heart) as described above.

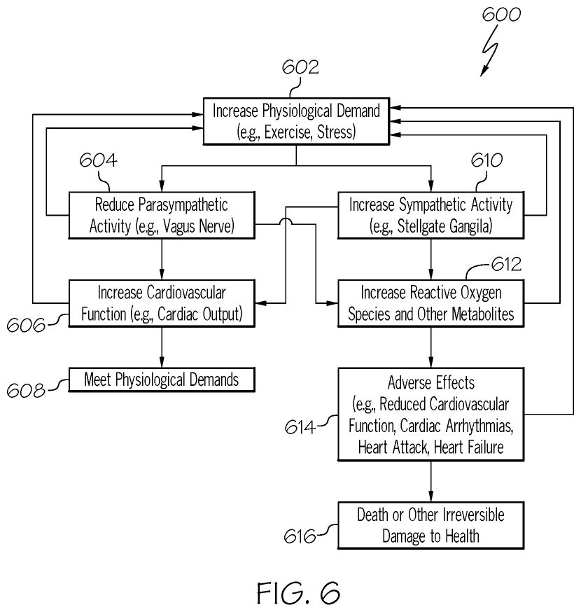

FIG. 6 depicts an example flow chart 600 including various steps for modulating sympathetic and parasympathetic nerves in order to reduce the risk of cardiac arrest while optimizing cardiac operation, according to one or more embodiments described and illustrated herein.

The steps illustrated in FIG. 6 relate to an automated and personalized method of modulating sympathetic and parasympathetic nerve activity (personalized titration of cardiac function and risk) in order to increase cardiac output in certain situations, while simultaneously reducing the risk of cardiac decompensation or sudden death. Certain conventional techniques for preventing sudden cardiac arrest or sudden death include performing a surgical excision of, e.g., stellate ganglia. While such a procedure may reduce the risk of sudden death, it may have the deficiency of adversely affecting the sympathetic drive required for individuals to perform various basic tasks. It is further noted that permanent activation or inhibition of the activity of the sympathetic nerve system (SNS) can have serious deleterious effects.

The implantable device 102 (e.g., a smart implantable neurophotonic device), as described in the present disclosure addresses and overcome the above described deficiency. Specifically, the implantable device 102, which operates in conjunction with the external control unit 114 to implement an automated and personalized nerve activity modulation method of the present disclosure, uses an artificial intelligence engine (e.g., AI neural network trained model) to modulate the activity or operation of the stellate ganglia. Such modulation reduces free radicals and the likelihood of sudden cardiac death, while simultaneously optimizing cardiac operation in individuals. Additionally, the external control unit 114 utilizes an artificial intelligence engine (which implements computationally efficient AI algorithms) to continuously assess the risk of sudden cardiac death along with various activities of an individual. Specifically, assessing the risk of sudden cardiac death includes detecting one or more of reactive oxygen species (ROS) levels, arrhythmias, ectopic heart beats, and blood pressure, and assessing various activities of the individual may include tracking of physiological data such as respiration rate, pulse rate, activity levels, metabolite levels, etc. Tracking or detection of other body signals (e.g., ECG, PPG, temperature, free radicals, etc.) across various sub-groups (e.g., based on gender, etc.) is also contemplated.

After tracking and detection of the above described data, the external control unit 114 may utilize an artificial intelligence engine to activate or inhibit the operation of the sympathetic nervous system (SNS). Specifically, the external control unit 114 may, using the artificial intelligence engine, generate stimulation parameters for graded or gradual activation or inhibition of the sympathetic nervous system. The stimulation parameters may be generated based on analyzing this data and comparing the data with historical responses and events that may be stored in one or more databases associated with the external control unit 114.

The stimulation parameters, when implemented by the implantable device 102, will result in the illumination of one or more of a plurality of light components (LEDs), which serve to accurately modulate SNS activity in individuals. The light components are nanotechnology-based light emitting diodes. In embodiments, as stated above, genes may be transferred into the stellate ganglia in order to express new protein chimeras that respond to specific LEDs by activating or inhibiting the nerve cells of the SNS. In this way, the artificial intelligence engine may integrate, collate, and analyze cardiac arrest risk levels, physiological activity data, and so forth, to generate stimulation parameters that will then be utilized to effectively modulate SNS activity.

In FIG. 6, block 602 refers to increased physiological demand in an individual that may be due to exercise, stress, or other such activities. In embodiments, increased physiological demand may lead to increased sympathetic activity—the stellate ganglia (sympathetic nerves) may stimulate B-adrenergic receptors for adaptive positive inotropy (block 610). Such a step may result in increased reactive oxygen species levels and other metabolite levels (block 612), which in turn would lead to various adverse effects such as, e.g., reduced cardiovascular function, cardiac arrhythmias, heart attack, and heart failure (block 614). Additionally, such conditions may ultimately result in the death or irreversible damage to the health of an individual. It is noted, however, that the ROS levels within an individual will continuously be monitored to ensure that these levels do not result or approach levels that may result in sudden cardiac arrest or death. However, when the risk levels are low (e.g., risk of cardiac arrest is low, based on analysis of the ROS levels and other data), the external control device 114 may generate stimulation parameters for increased activation of the stellate ganglia. Specifically, the stellate ganglia activity may be increased when higher levels of physical activity and temperature levels indicate the need for an increase in cardiac function.

For example, if the risk of cardiac arrest are analyzed and deemed to be low, but it is determined that there is a need for increased cardiac activity or functionality, stimulation parameters may be generated for reducing parasympathetic activity (block 604) and increasing sympathetic activity (610), which will result in increased cardiovascular functioning (block 606). In this way, increased physiological demands (block 608) may be met and increased cardiac functioning may be achieved, all the while ensuring that the risk of cardiac arrest is monitored.

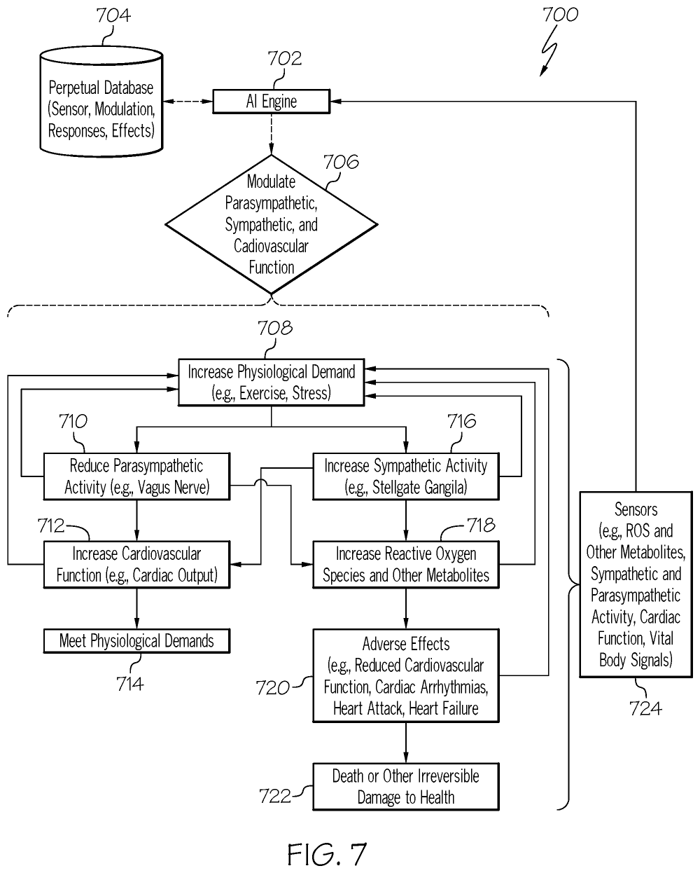

FIG. 7 depicts an example flow chart 700 including operation of an artificial intelligence engine that is utilized for modulating sympathetic and parasympathetic nerves in order to reduce the risk of cardiac arrest while optimizing cardiac operation, according to one or more embodiments described and illustrated herein.

At the outset, it is noted that blocks 708, 710, 712, 714, 716, 718, 720 and 722 correspond to blocks 602, 604, 606, 608, 610, 612, 614, and 616 in FIG. 6. As such, the description above in relation to blocks 602, 604, 606, 608, 610, 612, 614, and 616 apply to blocks 708, 710, 712, 714, 716, 718, 720 and 722 as well. Additionally, FIG. 7 depicts an artificial intelligence engine (block 702), that may be utilized by the external control device 114, to modulate parasympathetic, sympathetic, and cardio vascular functioning or operation based on data collected by the sensor 106. Specifically, the sensor 106 may gather data relating to ROS levels, metabolite levels, etc. Additionally, the modulation may also be based on various body signals such as, e.g., ECG, PPG, activity, temperature, free radical levels, etc.

In an example operation, the artificial intelligence engine of the external control device 114 may gather and integrate data that is obtained from the sensor 106, the stimulator 104, and one or more databases and modulate operation of the stellate ganglia in real time. Specifically, in an example operation, the external control device 114 utilizes an artificial intelligence engine to continuously monitor, in real time, body signals, CV functions, ROS and metabolite levels, and the risk or instances of various adverse health effects. The artificial intelligence engine may also analyze this information and compare this information with historical responses and events in order to generate stimulation parameters that are tailored to an individual and certain subgroups (e.g., gender, etc.). As stated, these stimulation parameters, when implemented by the implantable device 102, may result in modulation of the sympathetic and parasympathetic nerves and regulation or optimization in the functioning of the cardiovascular system of an individual. Such regulation or optimization may be in order to satisfy the physiological demands of an individual that is specific to a particular situation (e.g., combat conditions) while simultaneously preventing autonomic dysfunction, ROS overload, or other high risk of the occurrence of adverse events, e.g., cardiac arrest, sudden cardiac death, and so forth. It is further noted that, after modulation of the sympathetic and parasympathetic nerves, a response of an individual to the administered modulation may be stored and compared with historical data that catalogues various outcomes related to this individual and other individuals, and certain subgroups (e.g., gender, etc.). This information will then be utilized by the artificial intelligence engine to generate stimulation parameters that are tailored to an individual (i.e. stimulations parameters may be tailored specifically for the individual).

It is also noted that the artificial intelligence engine, as described above, continuously learns from historical and real time records of monitoring and modulating SNS activity, e.g., from an individual and/or across similar groups of individuals. Based on this learning, the external control unit 114 utilizes the artificial intelligence engine to generate stimulation parameters that are tailored to the individual and specific to a situation.

It should be understood that the embodiments described herein relate to a method for monitoring of cardiac-arrest risk levels and physiological activity of an individual and modulating sympathetic nerve activity based on the cardiac-arrest risk levels and the physiological activity. The method is implemented by a computing device and comprises receiving, in real time, cardiac risk data associated with the individual, the cardiac risk data, receiving, in real time, physiological data associated with the individual, analyzing using an artificial intelligence engine, in real time, the cardiac risk data and the physiological data associated with the individual, determining based on the analyzing, in real time, a cardiac risk level of the individual and a physiological activity level of the individual, generating first stimulation parameters for reducing the sympathetic nerve activity responsive to determining that the cardiac risk level exceeds a cardiac-risk metric, generating second stimulation parameters for increasing sympathetic nerve activity responsive based on: determining, using the artificial intelligence engine, that the physiological activity level of the individual satisfies a physiological activity metric, and determining that the cardiac risk level does not exceed a cardiac risk metric; and transmitting at least one of the first stimulation parameters and the second stimulation parameters to an implantable device.

It should also be understood that the embodiments described herein relate to a system for modulating operation of an organ in real time by controlling illumination of one or more light components is provided. The system includes an external device comprising a processing unit and a power supply configured to transmit stimulation parameters, a wireless implantable device comprising, a sensor configured to detect, in real time, activity data from a tissue cluster of an organ, a stimulator including a plurality of light components corresponding to at least a first wavelength and a second wavelength and a flexible elastomer coupled to the plurality of light components, and a transceiver configured to transmit the activity data to the external device, wherein the stimulator is configured to illuminate, based on the stimulation parameters, one of the plurality of light components coupled to the flexible elastomer, wherein the processing unit is configured to update the stimulation parameters based on the activity data.

The terminology used herein is for the purpose of describing particular aspects only and is not intended to be limiting. As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A system comprising:
an external device comprising a processing unit and a power supply configured to transmit stimulation parameters;
a wireless implantable device comprising:
a sensor configured to detect, in real time, activity data from a tissue cluster of an organ, wherein the activity data comprises a quantity of reactive oxygen species of the organ and the sensor detects an auto-fluorescence value from the tissue cluster of the organ, wherein the auto-fluorescence value is usable for determining the quantity of reactive oxygen species of the organ;
a stimulator including a plurality of light components corresponding to at least a first wavelength and a second wavelength and a flexible elastomer coupled to the plurality of light components; and
a transceiver configured to transmit the activity data to the external device,
wherein the stimulator is configured to illuminate, based on the stimulation parameters, one of the plurality of light components coupled to the flexible elastomer, and
wherein the processing unit is configured to update the stimulation parameters based on the activity data.

2. (canceled)

3. (canceled)

4. The wireless implantable device of claim 1, wherein the sensor detecting, in real time, the activity data from the tissue cluster of the organ includes the sensor detecting an additional auto-fluorescence value of at least one of a plurality of molecules.

5. The wireless implantable device of claim 4, wherein the plurality of molecules include proteins, arachidonic acid, and flavins.

6. The wireless implantable device of claim 1, wherein one of the plurality of light components includes a light emitting diode that is configured to emit a first light.

7. The wireless implantable device of claim 6, wherein the first light corresponds to a wavelength of 488 nanometers.

8. The wireless implantable device of claim 1, wherein another one of the plurality of light components includes an additional light emitting diode that is configured to emit a second light.

9. The wireless implantable device of claim 8, wherein the second light corresponds to a wavelength of 405 nanometers.

10. The wireless implantable device of claim 1, wherein the plurality of light components being coupled to the flexible elastomer includes a first light component embedded in a first portion of the flexible elastomer and at least a second light component embedded in a second portion of the flexible elastomer.

11. The wireless implantable device of claim 1, wherein the flexible elastomer is configured to attach to the tissue cluster of the organ.

12. A method comprising:
detecting, in real time, activity data from a tissue cluster of an organ, wherein the activity data comprises a quantity of reactive oxygen species of the organ and the sensor detects an auto-fluorescence value from the tissue cluster of the organ, wherein the auto-fluorescence value is usable for determining the quantity of reactive oxygen species of the organ;
transmitting the activity data to an external device;
receiving, from the external device, stimulation parameters for illumination of at least one of a plurality of light components, the plurality of light components coupled to a flexible elastomer; and
illuminating, based on the stimulation parameters, at least one of the plurality of light components.

13. (canceled)

14. (canceled)

15. The method of claim 14412, wherein the detecting, in real time, of the activity data from the tissue cluster of the organ includes detecting, in real time, an additional auto-fluorescence value of at least one of a plurality of molecules, the plurality of molecules including proteins, arachidonic acid, and flavins.

16. The method of claim 12, wherein one of the plurality of light components includes a light emitting diode that is configured to emit a first light.

17. The method of claim 16, wherein the first light corresponds to a wavelength of 488 nanometers.

18. A method for monitoring of cardiac-arrest risk levels and physiological activity of an individual and modulating sympathetic nerve activity based on the cardiac-arrest risk levels and the physiological activity, the method is implemented by a computing device, the method comprising:
receiving, in real time, cardiac risk data associated with the individual;
receiving, in real time, physiological data associated with the individual;
analyzing using an artificial intelligence engine, in real time, the cardiac risk data and the physiological data associated with the individual;
determining based on the analyzing, in real time, a cardiac risk level of the individual and a physiological activity level of the individual;
generating first stimulation parameters for reducing the sympathetic nerve activity responsive to determining that the cardiac risk level exceeds a cardiac-risk metric;
generating second stimulation parameters for increasing sympathetic nerve activity based on:
determining, using the artificial intelligence engine, that the physiological activity level of the individual satisfies a physiological activity metric, and
determining that the cardiac risk level does not exceed a cardiac risk metric; and
transmitting at least one of the first stimulation parameters and the second stimulation parameters to an implantable device.

19. The method of claim 18, wherein the implantable device is a neurophotonic device.

20. The method of claim 18, wherein the cardiac risk data includes one or more of reactive oxygen species levels, arrhythmias, ectopic heart beats, and blood pressure and the physiological data includes respiration rate, pulse rate, activity levels, and metabolite levels.
